(19) United States
(12) Patent Application Publication
(10) Pub. No.: US 2004/0082667 A1
(43) Pub. Date: Apr. 29, 2004

(54) Hangover treatment

(76) Inventors: Kenneth McCadden, Carle Place, NY (US); Grace MaCadden, Carle Place, NY (US)

Correspondence Address: COLLAR & ROE, P.C., 1077 Northern Boulevard, Roslyn, NY, 11576-1696, US

(21) Appl. No.: 10/686,415

(22) Filed: Oct. 15, 2003

Related U.S. Application Data
(60) Provisional application No. 60/418,777, filed on Oct. 16, 2002.

(51) Int. Cl. A61K 31/10

(52) U.S. Cl. 514/709

(57) ABSTRACT

A method of treating and preventing an alcohol induced hangover. A therapeutically effective amount of methylsulfonylmethane is adminsitered to a person prior to or after the person has consumed alcoholic beverages. The therapeutically effective amount of methylsulfonylmethane may be approximately 0.06 ounces of substantially pure methylsulfonylmethane in powder form. The methylsulfonylmethane may be mixed with a liquid such as water or juice or administered as a solid oral dosage form such as a tablet or capsule.

### CROSS REFERENCE TO RELATED APPLICATIONS

This application claims the benefit of U.S. Provisional Application No. 60/418,777 filed Oct. 16, 2002

### BACKGROUND OF THE INVENTION

1. Field of the Invention

This invention relates to the treatment and prevention of alcohol induced hangovers by the administration of the compound methylsulfonylmethane.

2. The Prior Art

The term “alcohol” as used herein refers to ethyl alcohol and “alcoholic-beverages” refers to popular spirits or blends that are intended for human consumption.

The social and economic consequences of alcohol overindulgence are well documented. Studies have indicated that approximately 1400 college students die each year as a result of unintentional alcohol-related injuries, including motor vehicle collisions, and 500,000 college students are unintentionally injured each year in alcohol-related incidents. (Hingson, R. W.; Heeren, T.; Zakocs, R. C.; et al. Magnitude of alcohol-related mortality and morbidity among U.S. college students ages 18-24. Journal of Studies on Alcohol 63(2), 2002). Alcohol use among college students has been linked to assault, sexual abuse, vandalism and other criminal activity . (Hingson et al.; Wechsler H., Lee J. E., Kuo M., Seibring M., Nelson T. F., Lee H. P., Trends in college binge drinking during a period of increased prevention efforts: Findings from four Harvard School of Public Health study surveys, 1993-2001. Journal of American College Health 50(5), 2002). Additionally, alcohol use and abuse is related to health and academic problems among college students. (Hingson et al., Wechsler et al.).

The after effects of over-consumption of alcoholic beverages are well-known and are manifested in symptoms which include headache, nausea, dizziness, loss of appetite, dehydration and body aches. These symptoms are commonly referred to, collectively and individually, as a hangover.

There are many products available which are claimed to be effective in the treatment of alcohol induced hangovers. They include aspirin as well as various natural ingredients and combinations of natural ingredients which are to be ingested before, during and/or after alcohol consumption. Additionally there are known many “folk” hangover cures from around the world, for example consuming a large T-bone steak or a chocolate milk shake (Australia); consuming thick, hot onion soup (France); rubbing a lemon under the arm used for drinking (Puerto Rico); consuming heavily salted cucumber juice or black bread soaked in water (Russia); and consuming a sip of brandy with a dash of peppermint (Switzerland).

Methylsulfonylmethane, also known as MSM or organic sulfur, is a naturally occurring sulfur compound found within the human body. It is not a sulfate or a sulfite. The toxicity level of methylsulfonylmethane is similar to water and is 1/7 that of common table salt.

Methylsulfonylmethane is stored in the cells of the human body and of the minerals present in the body, methylsulfonylmethane ranks third in quantity. The body uses approximately ⅛ of a teaspoon of methylsulfonylmethane each day. Excess methylsulfonylmethane is not stored by the body but rather is excreted in the manner of vitamin C.

Methylsulfonylmethane is present naturally in foods and mammal's milk, however the amount of methylsulfonylmethane consumed by humans is depleted as a result of food processing. Methylsulfonylmethane has been used in the treatment of arthritis, osteoarthritis, lupus, bursitis, gout and kidney stones. Published patents concerning the use of methylsulfonylmethane to prevent, treat or relieve various conditions include: U.S. Pat. Nos. 6,440,391 and 5,569,679 (management of snoring); U.S. Pat. No. 4,973,605 (relief of pain and nocturnal cramps and reduction of stress-induced death in animals); U.S. Pat. No. 4,568,547 (use as an excipient in tableting and encapsulating pharmaceutical dosage forms); U.S. Pat. No. 4,559,329 (reduction of gastric upset and allergic reactions); and U.S. Pat. No. 4,447,469 (as a topical preparation to soften skin, nails and other tissues).

### SUMMARY OF THE INVENTION

The invention relates to the treatment and prevention of alcohol induced hangovers by the administration of the compound methylsulfonylmethane. In a first embodiment, approximately 0.06 ounces of substantially pure methylsulfonylmethane in powdered form are mixed with approximately eight ounces of juice or water and administered to a person who is experiencing the symptoms of an alcohol-induced hangover. In another embodiment, a therapeutically effective amount of methylsulfonylmethane is incorporated into a solid oral dosage form, such as a tablet or capsule and administered to a person who is experiencing the symptoms of an alcohol-induced hangover.

### DETAILED DESCRIPTION OF THE PREFERRED EMBODIMENTS

Essentially, the invention relates to the treatment and prevention of an alcohol induced hangover by administering the compound methylsulfonylmethane. When a person ingests alcoholic beverages, the alcohol enters and remains in the cells of the body and prevents the uptake of oxygen by the cells. This results in the unpleasant feelings associated with an alcohol induced hangover. Methylsulfonylmethane causes the cell walls to become slippery and softer, allowing alcohol to be flushed out and oxygen to enter the cells and relieve the hangover symptoms.

In a first embodiment, approximately 0.06 ounces of substantially pure methylsulfonylmethane powder are mixed with approximately eight ounces of water or juice. The water may be plain or distilled. The mixture is then administered to a person who is experiencing the symptoms of an alcohol induced hangover. The after-effects of alcohol consumption, for example headache, nausea, dizziness, loss of appetite, dehydration and body aches are typically eliminated within approximately ½ hour of administration.

Substantially pure methylsulfonylmethane powder may be packaged in a packet, package or envelope known in the art. Each packet, package or envelope may comprise an individual dose, for example 0.06 ounces of substantially pure methylsulfonylmethane powder.

In a second embodiment, methylsulfonylmethane is administered to a person prior to that person's consumption of alcohol. The onset of symptoms associated with an alcohol induced hangover is thereby prevented.

In another embodiment, the methylsulfonylmethane compound is incorporated into a solid oral dosage form, such as a tablet or capsule, and ingested by a person who is experiencing the symptoms of an alcohol induced hangover.

When incorporated into a solid dosage form, such as a tablet or capsule, the solid dosage form may be packaged, for example in a bottle, blister pack, box, carton or any suitable packaging known in the art.

### EXPERIMENTAL RESULTS

Eight subjects (four male and four female) participated in a focus group to demonstrate the effectiveness of methylsulfonylmethane in treating an alcohol induced hangover. Each subject consumed an average of 9.5 alcoholic drinks in an average time period of 4.1 hours over the course of one evening. Each subject reported that they experienced the effects of an alcohol induced hangover the following morning. Upon rising, the subjects each ingested approximately 0.06 ounces of methylsulfonylmethane powder which had been mixed with approximately 8 ounces of water or juice. Each subject attested to the fact the effects of their alcohol induced hangover were relieved within less than one hour after ingesting the methylsulfonylmethane compound.

While several embodiments of the present invention have been described, it is obvious that many changes and modifications may be made thereunto without departing from the spirit and scope of the invention.

What is claimed:

1. A method of treating a person experiencing an alcohol induced hangover comprising administering a therapeutically effective amount of methylsulfonylmethane to the person experiencing an effect of an alcohol induced hangover.

2. The method according to claim 1, wherein said therapeutically effective amount of methylsulfonylmethane comprises approximately 0.06 ounces.

3. The method according to claim 1, wherein said therapeutically effective amount of methylsulfonylmethane is mixed with a liquid prior to said administering to the person experiencing an effect of an alcohol induced hangover.

4. The method according to claim 3 wherein said liquid comprises a water or a juice.

5. The method according to claim 1, wherein said therapeutically effective amount of methylsulfonylmethane is administered orally.

6. The method according to claim 5, wherein said therapeutically effective amount of methylsulfonylmethane is administered as a solid oral dosage form comprising a tablet or a capsule.

7. The method according to claim 6, further comprising the step of packaging said solid oral dosage form in a bottle, blister pack, box or carton.

8. The method according to claim 1, further comprising the step of packaging an amount of methylsulfonylmethane in a packet, package or envelope.

9. A method of preventing an alcohol induced hangover in a person comprising administering a therapeutically effective amount of methylsulfonylmethane to the person prior to the person's consumption of alcohol.

10. The method according to claim 9, wherein said therapeutically effective amount of methylsulfonylmethane comprises approximately 0.06 ounces.

11. The method according to claim 9, wherein said therapeutically effective amount of methylsulfonylmethane is mixed with a liquid prior to said administering to the person prior to the person's consumption of alcohol.

12. The method according to claim 11, wherein said liquid comprises a water or a juice.

13. The method according to claim 9, wherein said therapeutically effective amount of methylsulfonylmethane is administered orally.

14. The method according to claim 13, wherein said therapeutically effective amount of methylsulfonylmethane is administered as a solid oral dosage form comprising a tablet or a capsule.

15. The method according to claim 14, further comprising the step of packaging said solid oral dosage form in a bottle, blister pack, box or carton.

16. The method according to claim 9, further comprising the step of packaging an amount of methylsulfonylmethane in a packet, package or envelope.
